(19) United States
(12) Patent Application Publication
(10) Pub. No.: US 2005/0070965 A1
(43) Pub. Date: Mar. 31, 2005

(54) Method and system for discriminating RA driven from LA driven atrial flutter

(76) Inventors: Jong Kil, Glendale, CA (US); Xiaoyi Min, Thousand Oaks, CA (US); Peter Boileau, Valencia, CA (US)

Correspondence Address: PACESETTER, INC., 15900 VALLEY VIEW COURT, SYLMAR, CA, 91392-9221, US

(21) Appl. No.: 10/674,641

(22) Filed: Sep. 29, 2003

(51) Int. Cl. A61N 1/362

(52) U.S. Cl. 607/9

(57) ABSTRACT

A method and system for evaluating observed atrial activity, such as via an implantable cardiac stimulation device, to discriminate between the left and right atria as sites of origin of observed atrial flutter. The left and right atrial rates are compared for equivalence, stability, and relative time shift with respect to each other to determine which, if either, can be identified as the chamber of origin of the tachycardia. If relative comparison of the observed left and right atrial events is inconclusive, an evaluation can be made to attempt to determine the first flutter beat and its location under the assumption that the chamber in which a flutter beat was first observed was the site of origin. Determination of a particular atrium as the site of origin of flutter facilitates targeted delivery of ATP therapy to the determined site of origin.

### FIELD OF THE INVENTION

The invention is related to the field of implantable cardiac stimulation devices and, in particular, to methods and systems for discriminating atrial flutter driven from the left atrium vs. the right atrium and applying targeted therapy to the originating atrium.

### DESCRIPTION OF THE RELATED ART

Atrial flutter refers to a cardiac arrhythmia characterized by extremely rapid but generally regular atrial activity. Atrial flutter may be asymptomatic but frequently results in the afflicted person experiencing palpitations, weakness, etc. Flutter rhythm is generally in the range of approximately 250-350 beats per minute. The depolarization of atrial cells is generally coordinated in flutter leading to a typically stronger intracardiac electrogram/electrocardiogram (IEGM/ECG) signal strength than the generally unorganized fibrillation arrhythmia. However, the rate of flutter is too fast to allow efficient pumping action. Thus, a patient experiencing atrial flutter will have reduced efficiency in filling of the ventricles and, thus, reduced cardiac output.

A typical therapeutic intervention used to treat atrial flutter is referred to as antitachycardia pacing (ATP). ATP can be provided by an implantable cardiac stimulation device which typically uses standard bradycardia pacing algorithms and energy levels applied to the atria in an effort to bring the heart out of the tachycardia and restore a normal sinus rhythm. This is also sometimes referred to as ATP pacing.

If the re-entrant circuit is localized to one atrium, applied ATP therapy can be superfluous or pro-arrhythmic in the other atrium. As implantable cardiac stimulation devices are typically powered by a power supply having limited capacity, such as a battery, delivery of therapeutic stimulations where not needed needlessly depletes the limited battery capacity.

As atrial flutter reduces cardiac output and can be self-reinforcing, it is desirable to identify and terminate flutter as rapidly as possible. There is also a need to reduce unnecessary depletion of a battery with unneeded therapy. Thus, there is a need for methods and devices to more efficiently evaluate flutter and provide more efficient therapy.

### SUMMARY

It is accepted that the duration of atrial arrhythmia, such as atrial flutter, impacts the efficacy of atrial ATP. It is believed that the longer the patient is in atrial fibrillation/atrial flutter (AF/AFL), the more difficult it is for the therapy to convert the arrhythmia to a sinus rhythm. Thus, selecting the most effective ATP therapy earlier would be very important to the success of efficiently terminating the atrial arrhythmia. Finding the driving location of atrial flutter, i.e., whether the atrial flutter is driven from the right or left atrium, would help in selecting and prioritizing among a set of available ATP therapies by improving the ability to specifically target the driving location of the atrial arrhythmia.

With the availability of dual-site atrial leads in the right and left atria, IEGMs sensed at the right and atrial leads will provide information that may be used in discriminating the site of origin of atrial arrhythmias. One aspect of the invention is to distinguish right atrial driven from left atrial driven flutter by using cycle length/frequency differences resulting from left atrial driven flutter. Another aspect of the invention is to use observed timing differences of an initial flutter beat at right and left atrial sites to distinguish right atrial driven from left atrial driven flutter. Yet another aspect of the invention is to evaluate ongoing flutter beats at right and left atrial sites to distinguish between right atrial and left atrial driven flutter.

Two general ideas are implemented in the illustrated embodiments to determine the driving locations of atrial flutter. A first general idea implemented herein is to use the characteristics of the right and left atrial action potential durations implied through cycle length differences in the right and left atria when the left atrium is the driving source. The shorter duration of monophasic action potentials in the left atrium can sustain the reentrant waves at generally shorter cycle lengths than ones in the right atrium.

A second general idea implemented herein is to use timing differences of a first and/or ongoing flutter beats from two independent channels of pacing/sensing at right and left atrial sites. The timing differences are analyzed to determine an atrium of origin.

The aforementioned needs are satisfied and the above mentioned advantages are achieved by the invention which in one aspect is an implantable cardiac stimulation device that provides therapeutic electrical stimulation to the heart of a patient, including the left and right atrium, the device comprising a left atrail lead adapted to provide therapeutic stimulation to the left atrium, a right atrial lead adapted to provide therapeutic stimulation to the right atrium, at least one sensor that senses activity in the left and right atria and provides left and right atrial signals indicative thereof, and a processor that receives signals from the at least one sensor and induces therapeutic stimulation to be provided to at least one of the left and right atrium via the left atrium and right atrium leads respectively based at least in part on the signals received from the at least one sensor wherein, when the processor determines that both left and right atrium are experiencing atrial flutter, the processor evaluates the frequencies of the left and right atrial signals and, if one of the left and right signals has a higher frequency, the processor determines the atrium having the atrial flutter with the higher frequency to be the source of the atrial flutter and induces targeted delivery via the left or right atrial lead of an atrial flutter therapeutic stimulation to the atrium determined to be the source of atrial flutter.

In one embodiment, the device further comprises a memory in which the left and right atrial signals can be stored for subsequent evaluation by the processor to determine the source of origin of the atrial flutter and therein the processor can initially evaluate frequency and timing characteristics of the left and right atrial signals to determine the source of origin of the flutter event and, if neither the frequency nor timing indicates the source of origin, the processor evaluates the stored left and right atrial signals to determine which atrium signal exhibited an initial flutter event. The processor determines the atrium having the initial flutter event as the source of the atrial flutter and applies the atrial flutter therapeutic stimulation to that atrium.

In one embodiment, the frequencies of the left and right atrial signals are determined as the inverse of the interval between detected left and right atrial depolarizations. In one embodiment, the processor evaluates the respective frequencies of the left and right atrial signals and, if the respective frequency exceeds a pre-selected threshold, the processor determines that the respective atrium is in fibrillation and then induces the application of an atrial defibrillation therapeutic stimulation to that atrium.

In another embodiment, the processor evaluates the relative timing of the left and right atrial signals and, if the timing of one of the atrial signals precedes the other atrium signal by a threshold amount less than an interval between flutter events in one or more preceding cycles for a plurality of determined flutter events, then the processor determines the atrium corresponding to the atrium signal having the less preceding flutter events to be the source of the atrial flutter and induces the delivery of the targeted atrial flutter therapeutic stimulation to that atrium. Thereunder the threshold can comprise the flutter events of one atrium depolarization preceding the flutter event of the other atrium depolarization in a current cycle by an amount less than 40 percent of an interval between flutter events in one or more preceding cycles.

In one embodiment, the atrial flutter therapeutic stimulation comprises a plurality of successive electrical pulses applied via at least one of the left and right atrium leads. At least one of an amplitude, pulse width, interpulse interval, and number of pulses applied for the atrial flutter therapeutic stimulation can be programmable and/or the atrial flutter therapeutic stimulation can be applied synchronously with respect to at least one of the sensed left and right atrial signals. In one embodiment, the device further comprises at least one ventricle lead and at least one sensor that senses activity in at least one ventricle wherein the processor induces the delivery of appropriate therapeutic stimulation to at least one of the ventricles of the patient's heart.

Another embodiment is an implantable cardiac stimulation device that provides therapeutic electrical stimulation to the heart of a patient, including the left and right atrium, the device comprising at least one lead adapted to be implanted within at least one atrium of the patient so as to provide therapeutic stimulation, at least one sensor that senses activity in the left and right atria and provides left and right atrial signals indicative thereof, and a processor that receives signals from the at least one sensor and induces therapeutic stimulation to be provided to at least one atrium via the at least one lead based at least in part on the signals received from the at least one sensor wherein, when the processor determines that the left and atria are experiencing atrial flutter, the processor evaluates the frequencies of the left and right atrial signals and, if one of the left and right signals has a higher frequency, the processor determines the atrium having the atrial flutter with the higher frequency to be the source of the atrial flutter and induces targeted delivery of atrial flutter therapeutic stimulation to that atrium.

In this embodiment, at least one lead can comprise a left atrium lead and a right atrium lead and/or the at least one sensor can comprise a left atrium sensor and a right atrium sensor.

This embodiment can also further comprise a memory in which the left and right atrial signals can be stored for subsequent evaluation by the processor to determine the source of origin of the left and right atrial flutter and the processor can initially evaluate frequency and timing characteristics of the left and right atrial signals to determine the source of origin of the common flutter event and, if neither the frequency nor timing indicates the source of origin, the processor evaluates the stored left and right atrial signals to determine which atrium signal exhibited an initial flutter event and the processor determines the atrium having the initial flutter event as the source of the atrial flutter and applies the atrial flutter therapeutic stimulation to that atrium.

In one embodiment, the frequencies of the left and right atrial signals are determined as the inverse of the interval between detected left and right atrial depolarizations and/or the processor evaluates the respective frequencies of the left and right atrial signals and, if the respective frequency exceeds a pre-selected threshold, the processor determines that the respective atrium is in fibrillation and then induces the application of an atrial defibrillation therapeutic stimulation to the atrium experiencing the fibrillation.

In another embodiment, the processor evaluates the relative timing of the left and right atrial signals and, if the timing of one of the atrial signals precedes the other atrium signal by a threshold amount less than an interval between flutter events in one or more preceding cycles for a plurality of determined flutter events, then the processor determines the atrium corresponding to the atrium signal having the less preceding flutter events to be the source of the atrial flutter and induces the delivery of the targeted atrial flutter therapeutic stimulation to that atrium. Thereunder the threshold can comprise the flutter events of one atrium depolarization preceding the flutter event of the other atrium depolarization in a current cycle by an amount less than 40 percent of an interval between flutter events in one or more preceding cycles.

In a further embodiment, the atrial flutter therapeutic stimulation comprises a plurality of successive electrical pulses applied via the at least one lead. Therein at least one of an amplitude, pulse width, interpulse interval, and number of pulses applied for the atrial flutter therapeutic stimulation can be programmable and/or the atrial flutter therapeutic stimulation can be applied synchronously with respect to at least one of the sensed left and right atrial signals. In yet a further embodiment, the device further comprises at least one ventricle lead and at least one sensor that senses activity in at least one ventricle wherein the processor induces the delivery of appropriate therapeutic stimulation to at least one of the ventricles of the patient's heart.

An additional embodiment is an implantable cardiac stimulation device that provides therapeutic electrical stimulation to the heart of a patient, including the left and right atrium, the device comprising implantable means for sensing signals from and providing therapeutic stimulation to the left and the right atria and means for determining a source of origin of a combined atrial event wherein the determining means receives signals from and induces therapeutic stimulation to be provided by the sensing and stimulation means to at least one atrium based at least in part on the signals received from the sensing and stimulation means wherein, when the determining means determines that the left and right atria are experiencing atrial flutter, the determining means evaluates the frequencies of the left and right atrial signals and, if one of the left and right signals has a higher frequency, the determining means determines the atrium exhibiting the atrial flutter with the higher frequency to be the source of the atrial flutter and induces the sensing and stimulation means to delivery targeted atrial flutter therapeutic stimulation to that atrium.

The sensing and stimulation means can comprise electrodes implantable in the left and right atria and a pulse generator and/or the sensing and stimulation means can comprise at least one sensor implantable in the left and right atria and/or the determining means can comprise a programmable microprocessor.

In one embodiment, the device further comprises storage means in which the left and right atrial signals can be stored for subsequent evaluation to determine the source of origin of the left and right atrial flutter. The device can initially evaluate frequency and timing characteristics of the left and right atrial signals to determine the source of origin of the common flutter event and, if neither the frequency nor timing indicates the source of origin, the device evaluates the stored left and right atrial signals to determine which atrium signal exhibited an initial flutter event and determines the atrium having the initial flutter event as the source of the atrial flutter and applies the targeted atrial flutter therapeutic stimulation to that atrium.

In one embodiment, the device evaluates the respective frequencies of the left and right atrial signals and, if the respective frequency exceeds a pre-selected threshold, determines that the respective atrium is in fibrillation and then induces the application of an atrial defibrillation therapeutic stimulation to the atrium experiencing the fibrillation.

In another embodiment, the device evaluates the relative timing of the left and right atrial signals and, if the timing of one of the atrial signals precedes the other atrium signal by a threshold amount less than an interval between flutter events for one or more preceding cycles for a plurality of determined flutter events, the device determines the atrium corresponding to the atrium signal having the less preceding flutter events to be the source of the atrial flutter and induces the targeted delivery of the atrial flutter therapeutic stimulation to that atrium. The threshold can comprise the flutter events of one atrium depolarization preceding the flutter event of the other atrium depolarization in a current cycle by an amount less than 40 percent of an interval between flutter events in one or more preceding cycles.

In one embodiment, the atrial flutter therapeutic stimulation comprises a plurality of successive electrical pulses. Thereunder at least one of an amplitude, pulse width, interpulse interval, and number of the pulses applied for the atrial flutter therapeutic stimulation can be programmable and/or the atrial flutter therapeutic stimulation can be applied synchronously with respect to at least one sensed left and right atrium signal. In one embodiment, the device further comprises means for sensing activity in at least one ventricle and providing therapeutic stimulation to at least one ventricle.

Yet another embodiment is a method of determining the source of origin of a combined atrial flutter event affecting both the left and right atrium of a patient's heart using an implantable device, the method comprising evaluating flutter events of a flutter signal for both the left and right atria to determine at least one of the frequencies, relative timing, and initiation of the flutter signals and determining that the flutter signal having the highest frequency indicates that the corresponding atrium is the source of origin of the atrial flutter.

This method can further comprise determining whether the frequency of the flutter signals in the left and right atria is substantially equal and, if the frequency of the flutter signals is substantially equal, evaluating the relative timing of the flutter events of the left and right flutter signals to determine if a delay between left and right atrial flutter events for a current cycle is less than a determined amount different than delays in one or more preceding cycles to determine the origin of flutter. The delay of the left atrial event to the right atrial event in the current cycle being less than 40% that of the preceding cycle can determine a left atrium origin. The method can also further comprise applying antitachycardia therapy solely to the origin of flutter upon determination of the origin of flutter.

An additional embodiment is a method of providing therapeutic cardiac stimulation to a patient, the method comprising sensing cardiac activity in the patient's left atrium (LA) and right atrium (RA), evaluating the sensed left and right atrial activity for an observed atrial rate indicative of a tachycardia condition, upon detecting a tachycardia condition, comparing the left and right atrial rates for a current interval and:
if the LA and RA rates are unequal and the rates are unstable, determine fibrillation and apply defibrillation therapy;
if the LA and RA rates are unequal and stable, determine a LA origin of flutter and apply targeted antitachycardia pacing (ATP) therapy to the LA;
if the LA and RA rates are approximately equal and delay of LA to RA observed events is a determined amount less than that of a previous interval, determine a LA origin of flutter and apply targeted ATP therapy to the LA;
if the LA and RA rates are approximately equal and delay of RA to LA observed events is the determined amount less than that of the previous interval, determine a RA origin of flutter and apply targeted ATP therapy to the RA; or otherwise apply general ATP therapy to both atria.

In one embodiment, the determined amount is 40%. In another embodiment, when the method determines tachycardia, but does not determine fibrillation or a LA or RA origin of flutter, the method further comprises evaluating the observed LA and RA rates for the previous interval and, if the tachycardia can be determined to have existed first in the LA or the RA, then apply targeted ATP therapy to the LA or RA respectively, otherwise apply general ATP therapy to both atria. In yet another embodiment, evaluation of the LA and RA rates for stability comprises calculating an arithmetic mean period between observed events for the previous interval and determining whether individual periods are within a determined percent of the mean period.

A further embodiment is an implantable cardiac stimulation device comprising a plurality of implantable sensing and stimulation electrodes in communication with cardiac tissue of the left atria (LA) and right atria (RA),an implantable pulse generator in communication with the stimulation electrodes, and a microcontroller in communication with the sensing electrodes so as to receive signals therefrom wherein the microcontroller automatically evaluates signals received from the sensing electrodes, determines indications of tachycardia and, upon detection of a tachycardia, automatically evaluates stability and relative frequency of signals from the LA and RA and attempts to determine an atrium of origin of atrial flutter and, upon determination of an atrium of origin, induces the pulse generator to apply targeted antitachycardia pacing stimulation to stimulation electrodes in communication with the atrium of origin and, if flutter is determined but no determination of an atrium of origin is made, induces the pulse generator to apply antitachycardia pacing stimulation to stimulation electrodes in communication with both atria and if the tachycardia is determined to be fibrillation, induces the device to apply defibrillation therapy.

In one embodiment, the device distinguishes flutter from fibrillation according to the stability vs. instability respectively of observed LA and RA rates and/or determines the stability of observed LA and RA rates as the magnitude of the absolute differences of individual cardiac cycles from an arithmetic mean of a plurality of cardiac cycles as compared to a determined stability threshold.

In other embodiments, the device determines a LA origin of flutter if LA and RA rates are unequal and stable and/or determines a LA origin of flutter if LA and RA rates are approximately equal and delay of LA to RA observed events in a current cycle is a determined amount less than that of a previous cycle.

In further embodiments, the device determines a RA origin of flutter if the LA and RA rates are approximately equal and delay of RA to LA observed events is a determined amount less than that of a previous interval and/or determines the atrium of origin upon observation of tachycardia in the atrium of origin prior to tachycardia in the opposite non-origin atrium. These and other objects and advantages of the invention will be more apparent from the following description taken in conjunction with the accompanying drawings.

### BRIEF DESCRIPTION OF THE DRAWINGS

FIG. 1 is a simplified diagram illustrating an implantable stimulation device in electrical communication with at least three leads implanted into a patient's heart for delivering multi-chamber stimulation and shock therapy;

FIG. 2 is a functional block diagram of a multi-chamber implantable stimulation device illustrating the basic elements of a stimulation device which can provide cardioversion, defibrillation and pacing stimulation in four chambers of the heart;

FIG. 3 is a flow chart illustrating an embodiment of an implantable device sensing, storing, and evaluating atrial depolarizations signals for tachycardia conditions;

FIG. 4 is a flow chart showing an embodiment of discriminating LA and RA driven atrial flutter;

FIG. 5 is a more detailed embodiment of one of the determinations of the embodiment illustrated in FIG. 4;

FIG. 6 is a waveform showing sensed activity along a Bachmann's bundle showing a left-to-right directionality of impulse propagation;

FIG. 7 is a graph of LA rate vs. RA rate over regions indicative of normal sinus rhythm, flutter, and fibrillation indicating embodiments of the invention over various observed cardiac activity ranges; and

FIG. 8 is a sample ECG waveform illustrating onset of an atrial flutter condition, applied ATP therapy provided by the implantable device according to embodiments of the invention, and a return to sinus rhythm.

### DETAILED DESCRIPTION OF THE PREFERRED EMBODIMENT

The following description is of the best mode presently contemplated for practicing the invention. This description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout.

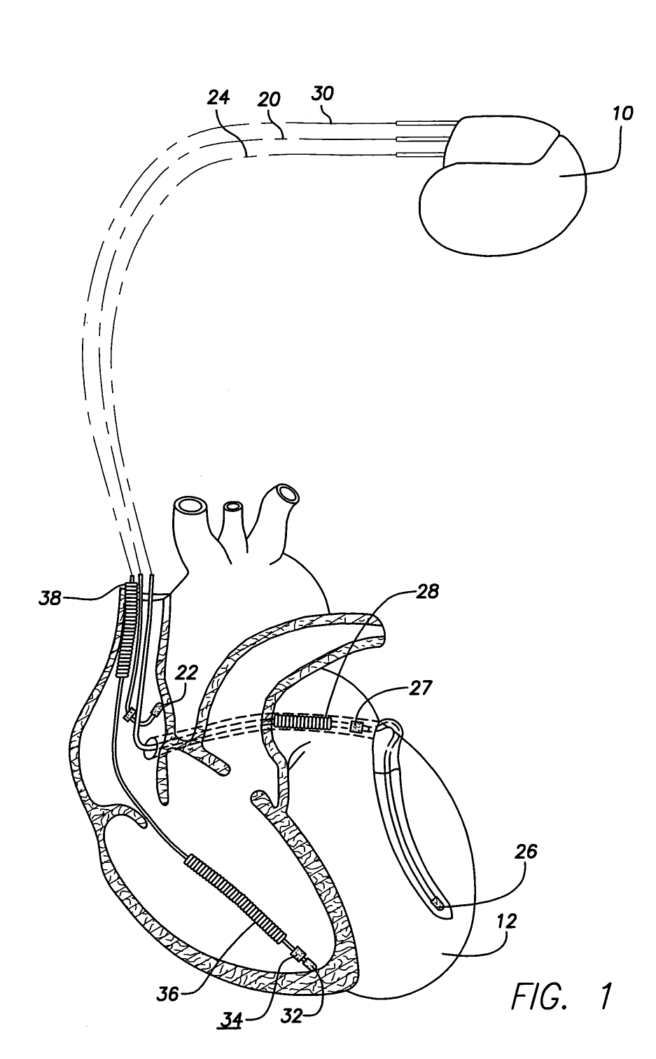

As shown in FIG. 1, there is an implantable stimulation device 10, referred to hereafter as “device 10” for brevity, in electrical communication with a patient's heart 12 by way of three leads, 20, 24 and 30, suitable for delivering multi-chamber stimulation and shock therapy. To sense atrial cardiac signals and to provide right atrial chamber stimulation therapy, the stimulation device 10 is coupled to an implantable right atrial lead 20 having at least an atrial tip electrode 22, which typically is implanted in the patient's right atrial appendage.

To sense left atrial and ventricular cardiac signals and to provide left chamber pacing therapy, the stimulation device 10 is coupled to a “coronary sinus” lead 24 designed for placement in the “coronary sinus region” via the coronary sinus ostium (OS) for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28.

The stimulation device 10 is also shown in electrical communication with the patient's heart 12 by way of an implantable right ventricular lead 30 having, in this embodiment, a right ventricular tip electrode 32, a right ventricular ring electrode 34, a right ventricular (RV) coil electrode 36, and an superior vena cava (SVC) coil electrode 38. Typically, the right ventricular lead 30 is transvenously inserted into the heart 12 so as to place the right ventricular tip electrode 32 in the right ventricular apex so that the RV coil electrode 36 will be positioned in the right ventricle and the SVC coil electrode 38 will be positioned in the superior vena cava. Accordingly, the right ventricular lead 30 is capable of receiving cardiac signals, and delivering stimulation in the form of pacing and shock therapy to the right ventricle.

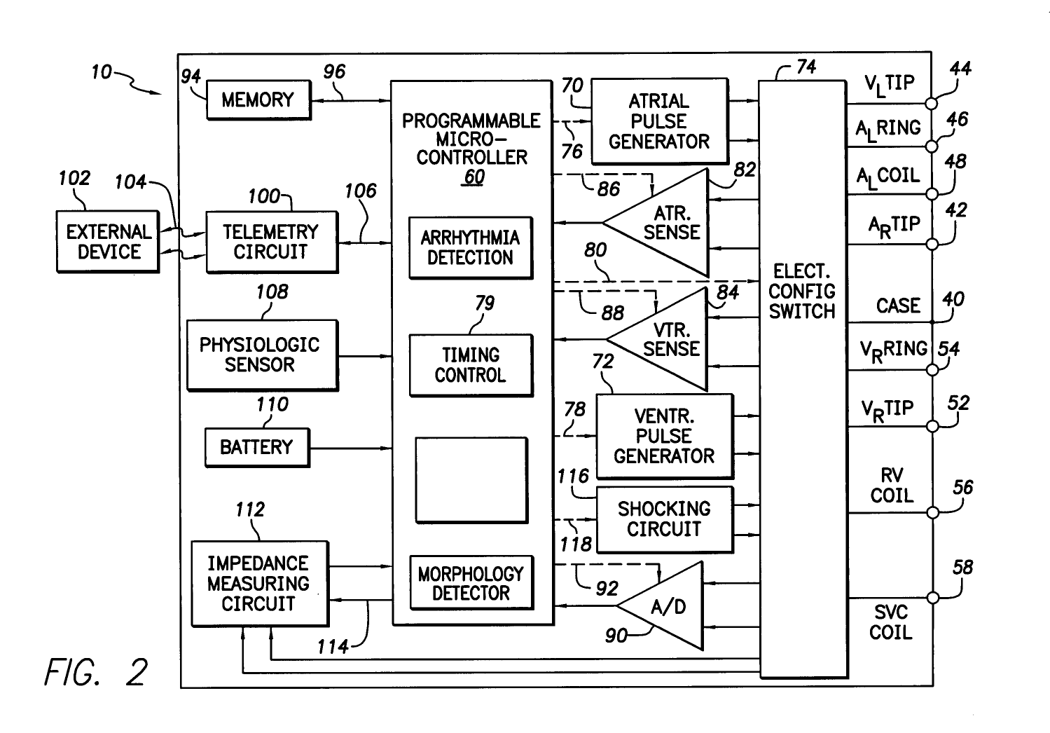

As illustrated in FIG. 2, a simplified block diagram is shown of the multi-chamber implantable stimulation device 10, which is capable of treating both fast and slow arrhythmias with stimulation therapy, including cardioversion, defibrillation, and pacing stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

A housing 40 for the stimulation device 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes, 28, 36 and 38, for shocking purposes. The housing 40 further includes a connector (not shown) having a plurality of terminals, 42, 44, 46, 48, 52, 54, 56, and 58 (shown schematically and, for convenience, the names of the electrodes to which they are connected are shown next to the terminals). As such, to achieve right atrial sensing and pacing, the connector includes at least a right atrial tip terminal (A_{R} TIP) 42 adapted for connection to the atrial tip electrode 22.

To achieve left chamber sensing, pacing and shocking, the connector includes at least a left ventricular tip terminal (V_{L} TIP) 44, a left atrial ring terminal (A_{L} RING) 46, and a left atrial shocking terminal (A_{L} COIL) 48, which are adapted for connection to the left ventricular tip electrode 26, the left atrial ring electrode 27, and the left atrial coil electrode 28, respectively.

To support right chamber sensing, pacing and shocking, the connector further includes a right ventricular tip terminal (V_{R} TIP) 52, a right ventricular ring terminal (V_{R} RING) 54, a right ventricular shocking terminal (R_{V} COIL) 56, and an SVC shocking terminal (SVC COIL) 58, which are adapted for connection to the right ventricular tip electrode 32, right ventricular ring electrode 34, the RV coil electrode 36, and the SVC coil electrode 38, respectively.

At the core of the stimulation device 10 is a programmable microcontroller 60 which controls the various modes of stimulation therapy. As is well known in the art, the microcontroller 60 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the present invention. Rather, any suitable microcontroller 60 may be used that carries out the functions described herein. The use of microprocessor-based control circuits for performing timing and data analysis functions are well known in the art.

As shown in FIG. 2, an atrial pulse generator 70 and a ventricular pulse generator 72 generate pacing stimulation pulses for delivery by the right atrial lead 20, the right ventricular lead 30, and/or the coronary sinus lead 24 via an electrode configuration switch 74. It is understood that in order to provide stimulation therapy in each of the four chambers of the heart 12, the atrial and ventricular pulse generators, 70 and 72, may include dedicated, independent pulse generators, multiplexed pulse generators, or shared pulse generators. The pulse generators, 70 and 72, are controlled by the microcontroller 60 via appropriate control signals, 76 and 78, respectively, to trigger or inhibit the stimulation pulses.

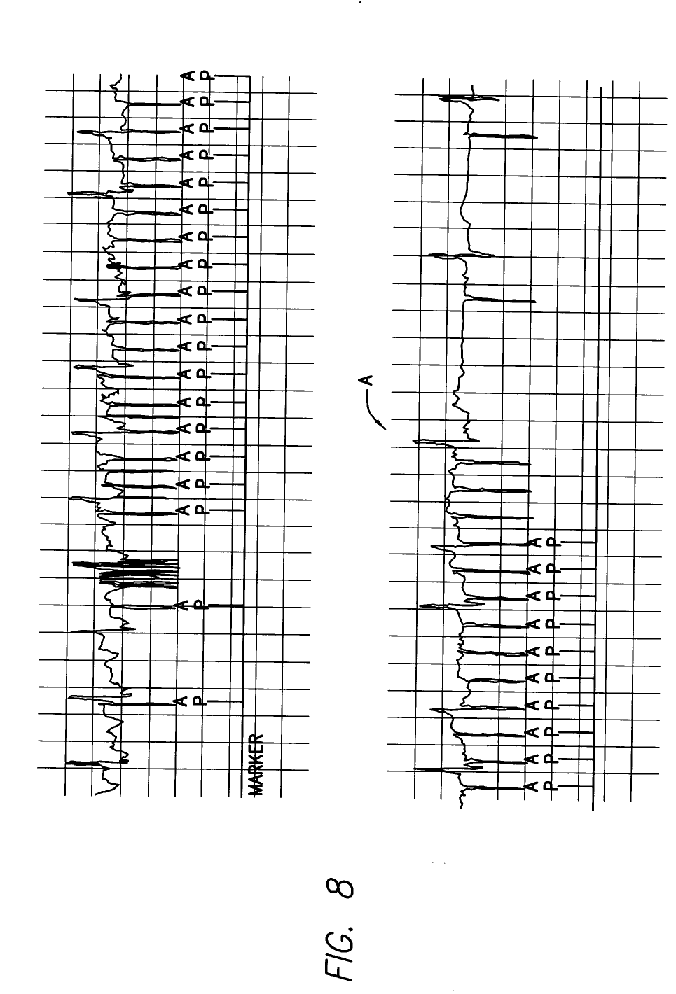

The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, PVARP intervals, noise detection windows, evoked response windows, alert intervals, marker channel timing, etc., which is well known in the art. The stimulation therapy provided by the device 10 according to aspects of the invention will be described in greater detail below with reference to FIG. 8.

The switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art.

Atrial sensing circuits 82 and ventricular sensing circuits 84 may also be selectively coupled to the right atrial lead 20, coronary sinus lead 24, and the right ventricular lead 30, through the switch 74 for detecting the presence of cardiac activity in each of the four chambers of the heart. Accordingly, the atrial (ATR. SENSE) and ventricular (VTR. SENSE) sensing circuits, 82 and 84, may include dedicated sense amplifiers, multiplexed amplifiers, or shared amplifiers. The switch 74 determines the “sensing polarity” of the cardiac signal by selectively closing the appropriate switches, as is also known in the art. In this way, the clinician may program the sensing polarity independent of the stimulation polarity.

Each sensing circuit, 82 and 84, preferably employs one or more low power, precision amplifiers with programmable gain and/or automatic gain control, bandpass filtering, and a threshold detection circuit, as known in the art, to selectively sense the cardiac signal of interest. The automatic gain control enables the device 10 to deal effectively with the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to the inputs of an analog-to-digital (A/D) data acquisition system 90. The data acquisition system 90 is configured to acquire intracardiac electrogram (IEGM) signals, convert the raw analog data into a digital signal, and store the digital signals for later processing and/or telemetric transmission to an external device 102, which, in certain embodiments, comprises a programmer. The data acquisition system 90 is coupled to the right atrial lead 20, the coronary sinus lead 24, and the right ventricular lead 30 through the switch 74 to sample cardiac signals across any pair of desired electrodes.

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 12 within each respective tier of therapy.

Advantageously, desired operating parameters or other programming instructions of the implantable device 10 may be non-invasively programmed into the memory 94 through a telemetry circuit 100 in telemetric communication with the external device 102, such as a programmer, transtelephonic transceiver, or a diagnostic system analyzer. The telemetry circuit 100 may be activated from a standby condition in response to an indication from a radio frequency (RF) detector (not shown) that signals of a predetermined strength are being received. The telemetry circuit 100 can communicate with the microcontroller 60 via a communication link 106.

The telemetry circuit 100 also advantageously allows intracardiac electrograms and status information relating to the operation of the device 10 (as contained in the microcontroller 60 or memory 94) to be sent to the external device 102 through an established communication link 104 as well as data from the sensor 108. In certain embodiments, data from the sensor 108 is selectively sent continuously via the communication link 104 and, in alternative embodiments, the data from the sensor 108 is sent in frames and/or as a derived signal, e.g. an average or rate.

The telemetry circuit 100 may advantageously operate at increased transmission rates. Increased data transmission rates of the telemetry circuit 100 enables the device 10 to transmit more data and/or data of increased detail than other devices. This aspect facilitates the display of additional information via the external device 102, such as a programmer.

The physiologic sensor 108 is commonly referred to as a “rate-responsive” sensor because it is typically used to adjust pacing stimulation rate according to the exercise state of the patient. However, the physiological sensor 108 may further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, AV Delay, V-V Delay, etc.) at which the atrial and ventricular pulse generators, 70 and 72, generate stimulation pulses.

While shown in FIG. 2 as being included internal to the stimulation device 10, it is to be understood that the physiologic sensor 108 may also be positioned outside and in communication with the stimulation device 10 and may include a variety of sensors 108 some or all of which may be external to the device 10, yet still be implanted within or carried by the patient. A common type of rate responsive sensor is an activity sensor, such as an accelerometer or a piezoelectric crystal, which is mounted within the housing 40 of the stimulation device 10. Other types of physiologic sensors are also known, for example, sensors which sense the oxygen content of blood, respiration rate and/or minute ventilation, pH of blood, ventricular gradient, etc. It is also to be understood, that in certain embodiments, the sensor 108 is capable of sensing multiple parameters and providing all the sensed parameters or a selected number of the parameters to the device 10.

The stimulation device additionally includes a battery 110 which provides operating power to all of the circuits shown in FIG. 2. For the stimulation device 10, which employs shocking therapy, the battery 110 must be capable of operating at low current drains for long periods of time, and then be capable of providing high-current pulses (for capacitor charging) when the patient requires a shock pulse. The battery 110 must also have a predictable discharge characteristic so that elective replacement time can be detected.

As further shown in FIG. 2, the device 10 is shown as having an impedance measuring circuit 112 which is enabled by the microcontroller 60 via a control signal 114. The known uses for an impedance measuring circuit 112 include, but are not limited to, lead impedance surveillance during the acute and chronic phases for proper lead positioning or dislodgment; detecting operable electrodes and automatically switching to an operable pair if dislodgment occurs; measuring respiration or minute ventilation; measuring thoracic impedance for determining shock thresholds; detecting when the device has been implanted; measuring stroke volume; and detecting the opening of heart valves, etc. The impedance measuring circuit 112 is advantageously coupled to the switch 74 so that any desired electrode may be used. The impedance measuring circuit 112 is not critical to the invention and is shown only for completeness.

In the case where the stimulation device 10 is intended to operate as an implantable cardioverter/defibrillator (ICD) device, it must detect the occurrence of an arrhythmia, and automatically apply an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 joules), as controlled by the microcontroller 60. Such shocking pulses are applied to the patient's heart 12 through at least two shocking electrodes and, as shown in this embodiment, selected from the left atrial coil electrode 28, the RV coil electrode 36, and/or the SVC coil electrode 38. As noted above, the housing 40 may act as an active electrode in combination with the RV electrode 36, or as part of a split electrical vector using the SVC coil electrode 38 or the left atrial coil electrode 28 (i.e., using the RV electrode as a common electrode).

Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

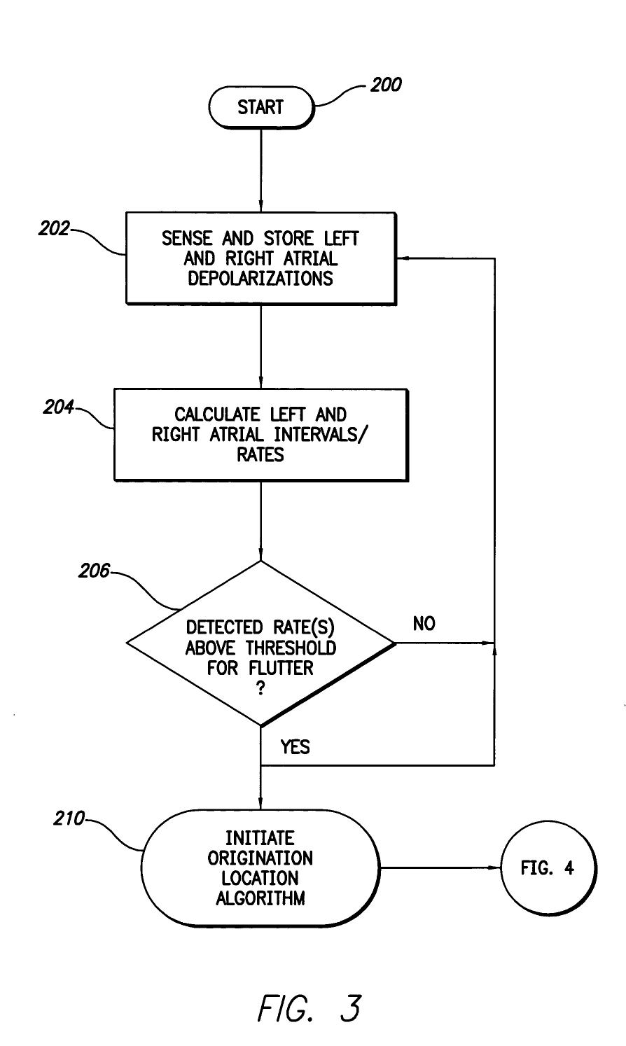

In FIGS. 3-5, flow chart are shown describing an overview of the operation and novel features implemented in one embodiment of the device 10. In these flow charts, the various algorithmic steps are summarized in individual “blocks”. Such blocks describe specific actions or decisions that are made or carried out as the algorithm proceeds. Where a microcontroller (or equivalent) is employed, the flow charts presented herein provide the basis for a “control program” that may be used by such a microcontroller (or equivalent) to effectuate the desired control of the stimulation device. Those skilled in the art may readily write such a control program based on the flow charts and other descriptions presented herein.

FIG. 3 illustrates the beginning of this embodiment as the start state 200. Following is the state 202 wherein the device 10 senses cardiac activity, such as left and right atrial depolarizations via the coronary sinus lead 24 and left atrial ring electrode 27 and the right atrial lead 20 and the atrial tip electrode 22 as well as the atrial sensing circuits 82. The device 10 evaluates these sensed signals and when a cardiac event of interest is detected, the device 10 stores information related to the detected atrial activity in memory 94.

In state 204, the device 10 evaluates the detected right and left atrial activity and calculates left and right intervals/rates. The device 10 also makes a decision in state 206 whether the detected atrial activity in either or both of the atria exceeds a threshold value for flutter. If flutter is detected in either or both of the atrial in state 206, the device 10 initiates an algorithm to attempt to determine the site of origin of the flutter in state 210. Whether or not the device 10 detects flutter in state 206, the device 10 continues to sense, evaluate, and store information relating to left and right atrial activity in states 202, 204, and 206. The sensed activity under which the device 10 “detects” atrial activity as well as the threshold defining flutter comprises parameters that can be programmable and defined for an individual patient and changed over time to improve the efficacy of the performance of the device 10.

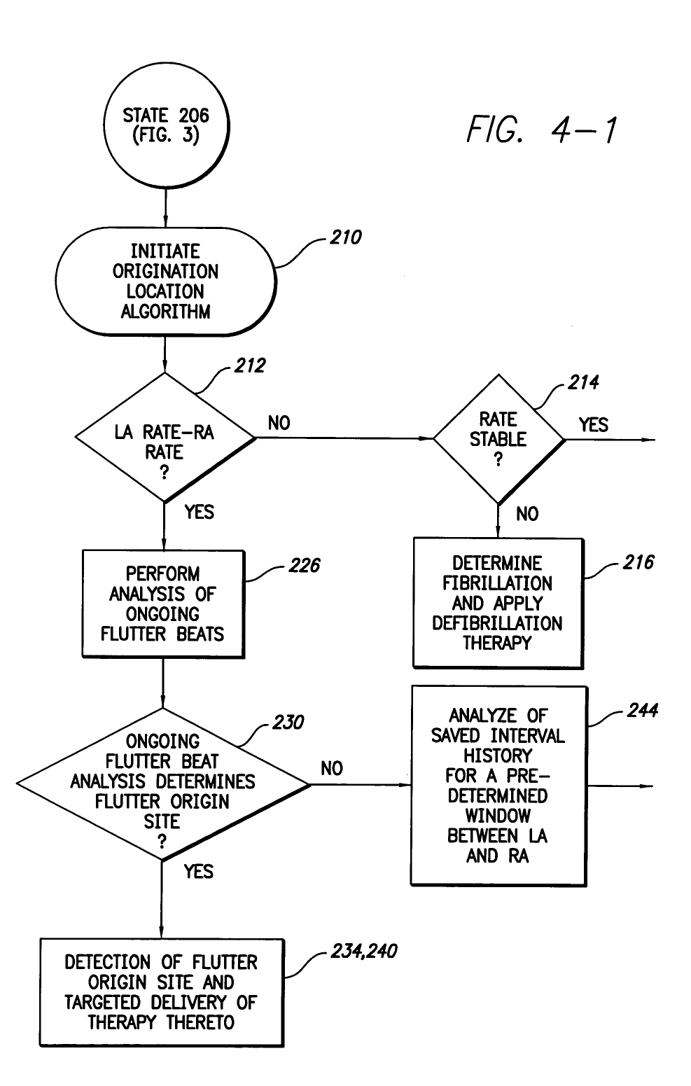

FIG. 4 is a flow chart illustrating a general overview of an embodiment of discriminating right atrial driven from left driven atrial flutter so as to provide targeted ATP therapy to the left or right atrium or to both. An evaluation is made in decision state 212 whether the observed left atrial rate is approximately equal to the right atrial rate. In one particular embodiment, cycle durations/intervals separately monitored in the right and left atria are evaluated using averaged cycle durations over a few cardiac cycles. The left and right durations/intervals correspond inversely to the left and right rates. The device 10 performs a comparison between the observed right and left cycle durations. If it is determined that the left atrial cycle lengths are unequal to the right atrial cycle lengths, the device 10 evaluates in state 214 the stability of the observed left and right atrial rates/intervals.

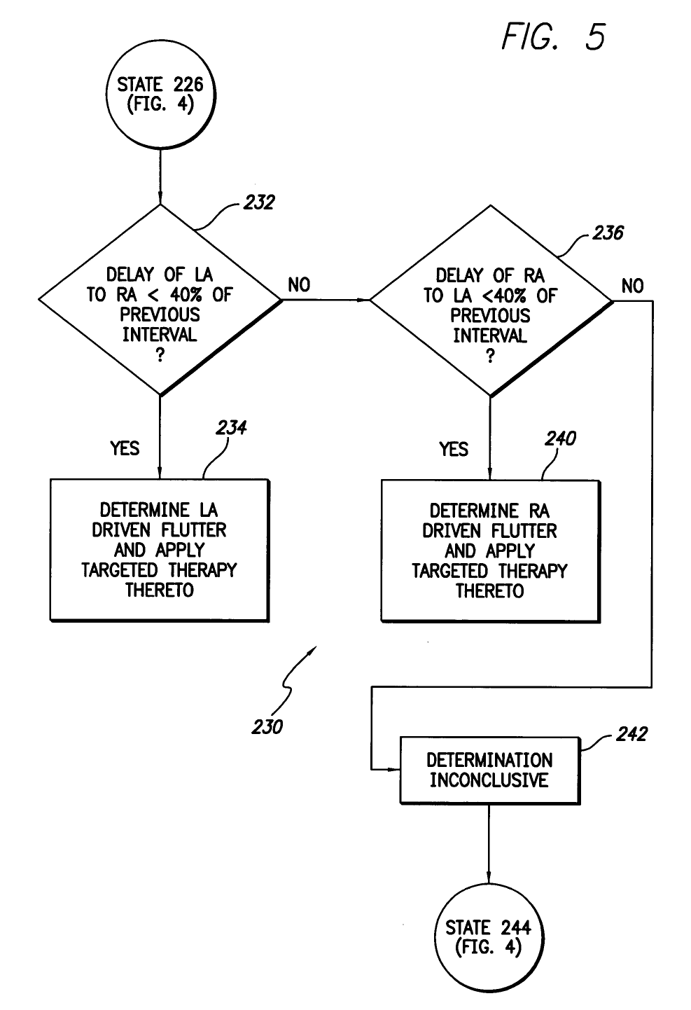

The stability of the LA and RA rates is a factor in discriminating flutter from fibrillation. One embodiment of evaluating stability is illustrated in FIG. 6. In this example, a number of waveforms are shown indicating observed cardiac activity at multiple sites across the heart, in this specific example three. The figure also illustrates a flutter condition with a spatial time gradient or delay in observed activity across the heart. FIG. 6 shows a plurality of observed time periods between observed cardiac events designated in this example as t_{F1}, t_{F2}, etc. for the observed periods at the site of flutter origin and t_{NF1}, t_{NF2}, etc. for the observed periods opposite the site of flutter origin.

These pluralities of observed time periods can be evaluated, for example, by evaluating the difference of individual observed time periods vs. an arithmetic mean time period. Stability of the observed rates can be made based upon the difference of individual periods being less than a determined percent of the observed mean or an absolute value less than the mean. Stability can also be determined as the percentage difference of the absolute value of individual periods vs. a mean period. Evaluation of the stability of the observed rates can also be also made, for example, in accordance with the teachings of U.S. Pat. No. 5,941,831 issued to Robert Turcott, Aug. 24, 1999, which is incorporated herein in its entirety by reference.

If the device 10 determines in state 214 that the atrial rates are not stable, the device 10 concludes that the rhythm is classified as atrial fibrillation and appropriate defibrillation shocking therapy would then be delivered as previously described in state 216.

If the device determines in state 214 that the atrial rates are stable, the device 10 determines in state 220 whether the left atrial (LA) rate is greater than the right atrial (RA) rate. If the LA rate is greater, the device 10 determines that the left atrium originating the flutter and the device applies targeted ATP therapy to the left atrium in state 222 and conversely, if the RA is greater, the device 10 determines that flutter is originating in the RA and applies targeted ATP therapy to the RA in state 224. The targeted ATP therapy provided in states 222 and 224 will be described in greater detail below with reference to FIG. 8.

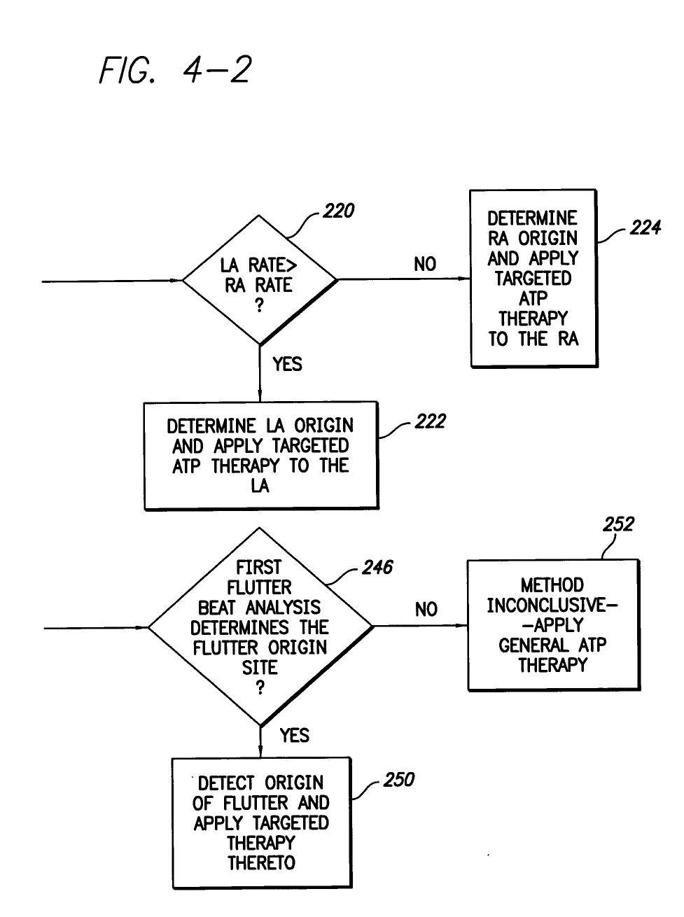

If the device 10 determines in state 212 that the left atrial rate is approximately equal to the right atrial rate, an analysis is performed of ongoing observed flutter beats in state 226 which leads to the decisions of state 230. The decisions of state 230 are described in greater detail with reference to FIGS. 5 and 6.

In particular, in the state 230, the device 10 performs an evaluation of observed atrial events such as are shown in FIG. 6. FIG. 6 illustrates observed atrial activity sensed, such as via the atrial tip 22 and left atrial ring 27 electrodes. FIG. 6 also indicates an example time shift in corresponding events progressing from the site of origin to the opposite atrium.

In state 230, delays between corresponding events in the left and right atria are compared with respect to the events observed in a previous cardiac cycle. The device 10 determines in state 232 if the delay between observed activity in the left atrium as compared to the right atrium is less than a determined amount than that observed in the previous interval, that the observed atrial flutter is driven from the left atrium and appropriately LA targeted ATP therapy is provided in a state 234. In one particular embodiment, the decision of state 232 is made under the condition that the delay of the left atrial to right atrial observed event is less than forty percent of that of the previous interval. It will be appreciated, however, that in other embodiments a different evaluation parameter can be used and that this parameter may be programmable in the device 10.

If the evaluation of state 232 is negative, i.e. that the delay of the left atrial observed activity to the observation of corresponding right atrial activity is not less than the determined criteria, a decision will be made in state 236 if the delay of observed right atrial activity to observed left atrial activity is less than a determined amount than that observed in the previous interval, in this example of forty percent. If the evaluation of state 236 is positive, the determination is made that the observed flutter is driven from the right atrium and appropriate targeted RA ATP therapy will be provided in the state 240.

If the results of both states 232 and 236 are negative, an evaluation will be made in state 242 that the determination of discriminating between driving origins in the left or right atrium is inconclusive at this stage. FIG. 6 illustrates this process graphically in that, assuming a common time scale, the plurality of wave forms shown in FIG. 6 are horizontally displaced and thus show a time gradient in the observed events considered between the left and right atria which the device 10 analyses to determine the atrium of origin.

State 242, e.g. a determination that the site of origin of the observed atrial flutter cannot yet be made leads to state 244. In state 244 an analysis is performed of past interval histories for a predetermined window between the left and right atria. In particular, the history of observed atrial events is evaluated to search for the occurrence of the very first flutter beat as illustrated in decision state 246. It is assumed herein that the stored interval will contain interval characteristics representative of a progression from normal sinus rhythm beats to the flutter beats and that the origin of the flutter is the site at which the very first flutter beat occurred, which is assumed to have occurred before observed flutter beats in the opposite chamber. One embodiment of this detection comprises an initial change in the interval between successive atrial depolarizations.

If a detectable first flutter beat can be determined along with a corresponding atrium, a decision will be reached in state 246 indicating a determination of the flutter origin and delivery of appropriately targeted ATP therapy to the atrium of origin in state 250. Alternatively, if the determination of decision state 244 is negative then the device 10 will determine that the discrimination between the left and right atria as sites of origin of the flutter is indeterminate in state 252 and appropriate global ATP is provided to both atria.

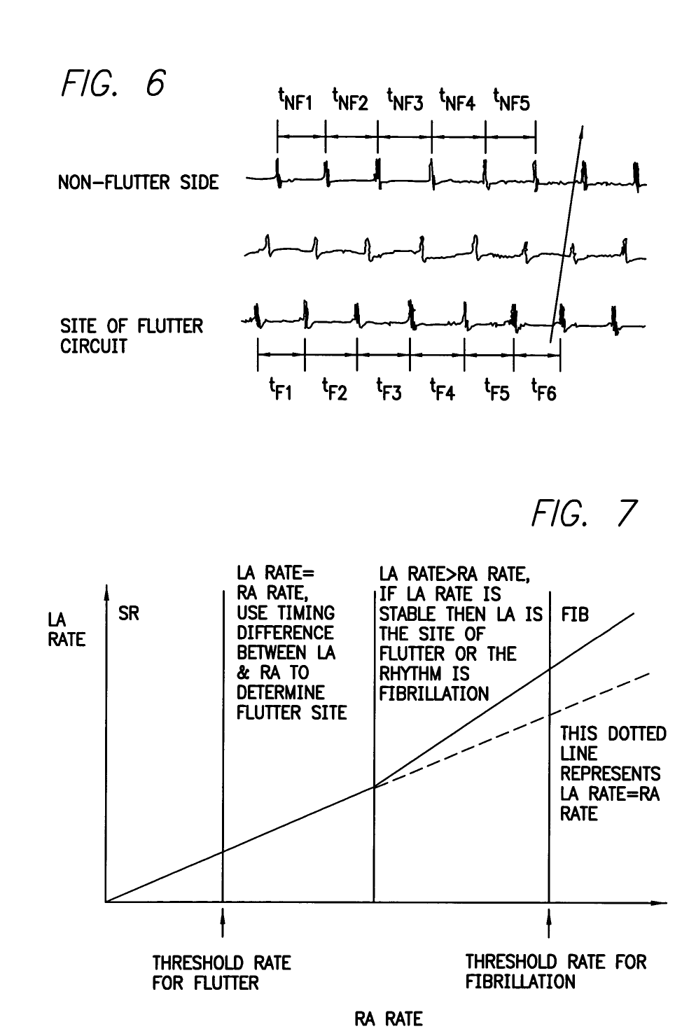

FIG. 7 graphically illustrates general relationships among possible expected observed left and right atrial rates and their evaluation in accordance with aspects of the invention. In particular, FIG. 7 shows the LA rate vs. the RA rate with a first region of LA rate approximately equal to the RA rate and below the threshold rate for flutter and thus indicating a sinus rhythm. Adjacent this region is shown a region where the RA and LA rates are approximately equal, but exceed the threshold for flutter. In this aspect of the invention, timing differences between the observed RA and LA activity are examined to determine the site of flutter origin.

The next region indicates yet higher RA and LA rates, but still below a threshold for fibrillation. This region also shows that the LA rate is greater than the RA rate. In this region, according to certain aspects of the invention, if the LA rate is stable, the LA is determined to be the site of origin of the flutter. This illustration assumes that left atrial driven flutter is expected to predominate, however right atrial driven flutter with corresponding greater RA rates can of course exist in other embodiments. The highest rate region indicates the RA and LA rates in excess of a threshold for fibrillation and, in this aspect of the invention, appropriate defibrillation therapy would be provided.

FIG. 8 illustrates a sample waveform of an electrocardiogram (ECG) of cardiac and stimulation therapy provided in accordance with embodiments of the invention and as observed at the surface of a patient's body via a plurality of surface electrodes. In this embodiment, the observed signals are shown with respect to the lead 11 configuration. The initial portion of the waveform illustrates an atrial flutter condition. In this example, a determination is made that the flutter originated in the right atrium in accordance with the aspects of the invention previously described.

ATP therapy targeted to the right atrium is then applied as illustrated with the marker trace of FIG. 8. In this embodiment, the targeted ATP therapy comprises a sequence of targeted pacing pulses applied to the right atrium via the right atrial lead 20 and the atrial tip electrode 22 as provided by the atrial pulse generator 70 of the device 10. In one embodiment, the pacing pulses comprise electrical stimulations of approximately 5 V applied at a pulse width of approximately 1 ms.

The device 10 applies ATP therapy in an attempt to overdrive the targeted atrium(atria) by capturing the excitable gap of the reentrant circuit. As such, the applied pacing pulses can be applied asynchronously or delivered with the first pulse of an ATP burst relative to a sensed atrial event. The timing of the initial pulse of an ATP burst relative to the sensed atrial event can comprise a delay of 0, a delay of a determined absolute value, or a delay comprising a percentage, such as 80%, of the sensed atrial interval. In one embodiment, the ATP burst is initiated with a 0 delay from a detected atrial event.

The ATP burst interpulse interval, in one embodiment, is determined as a percentage, in one example 80%, of the detected interval between atrial events previously detected. In one embodiment, the ATP interburst intervals include a ramp aspect wherein the interburst intervals progressively decrease (increasing the pulse rate) with time within each burst of ATP pulses. In alternative embodiments, the ATP interburst intervals also comprise a scan aspect wherein the intervals within a given burst are relatively constant, however the intervals are varied from burst to burst. In one embodiment, the intervals are shortened from burst to burst.

The exact parameters of pacing therapy applied by the device 10 are programmable for specific applications. Exemplary ranges for these parameters include 0.5-7 V pulse amplitude and pulse widths of 0.1-2 ms. In embodiments where the device 10 includes ICD functionality, the available voltage can extend to 10 or more V. The timing parameters, including delay from a sensed atrial event and interburst intervals both within a given burst and among multiple bursts, are also programmable.

The region of the ECG waveform indicated as “A” illustrates an effective result of the ATP therapy and a return to sinus rhythm. As previously described, the device 10 operating according to the embodiments of the invention described herein would continue to monitor the heart 12 for possible recurrence of a similar or other arrhythmia and provide indicated therapy.

Although the preferred embodiments of the present invention have shown, described and pointed out the fundamental novel features of the invention as applied to those embodiments, it will be understood that various omissions, substitutions and changes in the form of the detail of the device illustrated may be made by those skilled in the art without departing from the spirit of the present invention. Consequently, the scope of the invention should not be limited to the foregoing description but is to be defined by the appended claims.

1. An implantable cardiac stimulation device that provides therapeutic electrical stimulation to the heart of a patient, including a left atrium and a right atrium, the device comprising:
a left atrial lead adapted to be implanted within the patient so as to provide therapeutic stimulation to the left atrium;
a right atrial lead adapted to be implanted within the patient so as to provide therapeutic stimulation-to the right atrium;
one or more sensors that are collectively operative to sense activity associated with the left and right atria and to provide left and right atrial signals indicative thereof; and
a processor in electrical communication with the at least one sensor and that receives signals from the one or more sensors, wherein the processor is operative to evaluate frequencies of the left and right atrial signals and, if one of the left and right signals has a higher frequency, the processor determines the atrium with the higher frequency to be the source of the atrial flutter, wherein the processor is operative to control the pulse generator to initiate delivery of therapeutic stimulation via the left or right atrial lead to the atrium determined to be the source of atrial flutter.

2. The device of claim 1, further comprising a memory in which the left and right atrial signals can be stored for subsequent evaluation by the processor to determine the source of origin of the left and right atrial flutter.

3. The device of claim 2, wherein the processor initially evaluates frequency and timing characteristics of the left and right atrial signals to determine the source of origin of the common flutter event and, if neither the frequency nor timing indicates the source of origin, the processor evaluates the stored left and right atrial signals to determine which atrium signal exhibited an initial flutter event and the processor determines the atrium having the initial flutter event as the source of the atrial flutter and applies the atrial flutter therapeutic stimulation to that atrium.

4. The device of claim 1, wherein the frequencies of the left and right atrial signals are determined as the inverse of the interval between detected left and right atrial depolarizations.

5. The device of claim 1, wherein the processor evaluates the respective frequencies of the left and right atrial signals and, if the respective frequencies exceed a pre-selected threshold, the processor determines that the respective atrium is in fibrillation and then induces the application of an atrial defibrillation therapeutic stimulation to the heart.

6. The device of claim 1, wherein the processor evaluates the relative timing of the left and right atrial signals and, if the timing of one of the atrial signals precedes the other atrium signal by a threshold amount less than an interval between flutter events in one or more preceding cycles for a plurality of determined flutter events, then the processor determines the atrium corresponding to the atrium signal having the less preceding flutter events to be the source of the atrial flutter and induces the delivery of the targeted atrial flutter therapeutic stimulation to that atrium.

7. The device of claim 6, wherein the threshold comprises the flutter events of one atrium depolarization preceding the flutter event of the other atrium depolarization in a current cycle by an amount less than 40 percent of an interval between flutter events in one or more preceding cycles.

8. The device of claim 1, wherein the atrial flutter therapeutic stimulation comprises a plurality of successive electrical pulses applied via at least one of the left and right atrial leads.

9. The device of claim 8, wherein at least one of an amplitude, pulse width, interpulse interval, and number of pulses applied for the atrial flutter therapeutic stimulation is programmable.

10. The device of claim 8, wherein the atrial flutter therapeutic stimulation is applied synchronously with respect to at least one of the sensed left and right atrial signals.

11. The device of claim 1, further comprising at least one ventricle lead and at least one sensor that senses activity in at least one ventricle wherein the processor induces the delivery of appropriate therapeutic stimulation to at least one of the ventricles of the patient's heart.

12. An implantable cardiac stimulation device that provides therapeutic electrical stimulation to the heart of a patient, including a left atrium and a right atrium, the device comprising:
means for sensing signals from the left and the right atria;
means for determining a source of origin of a combined atrial event wherein the determining means receives signals from the means for sensing, wherein the means for determining comprises means for evaluating the frequencies of the left and right atrial signals to determine an originating atrium; and
means for delivering atrial flutter therapeutic stimulation to the originating atrium.

13. The device of claim 12, wherein the means for sensing comprises electrodes implantable in the patient's heart for communication with the left and right atria.

14. The device of claim 12, wherein the means for sensing comprises at least one sensor implantable in the patient's heart.

15. The device of claim 12, further comprising means for detecting fibrillation when the frequency exceeds a pre-selected threshold.

16. A method of determining the source of origin of an atrial flutter event using an implantable device, the method comprising:
evaluating flutter events of a flutter signal for both the left and right atria to determine the frequencies of the flutter signals; and
determining that the flutter signal having the highest frequency indicates that the corresponding atrium is the source of origin of the atrial flutter.

17. The method of claim 16 further comprising: determining whether the frequency of the flutter signals in the left and right atria is substantially equal; and if the frequency of the flutter signals is substantially equal, evaluating the relative timing of the flutter events of the left and right flutter signals to determine if a delay between left and right atrial flutter events for a current cycle is less than a determined amount different than delays in one or more preceding cycles to determine the origin of flutter.

18. An implantable cardiac stimulation device comprising:
a plurality of implantable sensing and stimulation electrodes in communication with cardiac tissue of the left atria (LA) and right atria (RA);
an implantable pulse generator in communication with the stimulation electrodes; and
a microcontroller in communication with the sensing electrodes so as to receive signals therefrom, wherein the microcontroller is operative to evaluate signals received from the sensing electrodes, determine indications of tachycardia and, upon detection of a tachycardia, automatically evaluate stability and relative frequency of signals from the LA and RA and attempts to determine an atrium of origin of atrial flutter and, upon determination of an atrium of origin, and controls the pulse generator to apply stimulation to the atrium of origin.

19. The device of claim 18, wherein the device determines a LA origin of flutter if LA and RA rates are unequal and stable.

20. The device of claim 18, wherein the device determines a RA origin of flutter if the LA and RA rates are approximately equal and delay of RA to LA observed events is a determined amount less than that of a previous interval.
